(19) United States
(12) Patent Application Publication
(10) Pub. No.: US 2004/0072225 A1
(43) Pub. Date: Apr. 15, 2004

(54) Microarray retrieval unit

(75) Inventors: Eric Rollins, Palo Alto, CA (US); Josh Guyot, Fly Creek, NY (US); JoeBen Bevirt, Emerald Hills, CA (US)

Correspondence Address: INCYTE CORPORATION, 3160 PORTER DRIVE, PALO ALTO, CA, 94304, US

(73) Assignee: Incyte Corporation, Palo Alto, CA (US)

(21) Appl. No.: 10/611,474

(22) Filed: Jun. 30, 2003

Related U.S. Application Data
(62) Division of application No. 09/639,732, filed on Aug. 15, 2000, now abandoned.

(51) Int. Cl. C12Q 1/68

(52) U.S. Cl. 435/6

(57) ABSTRACT

The present invention provides a retrieval unit for a microarray processing device to transport a workpiece such as a microscope slide before or after processing operations by the microarray processing device. The retrieval unit may include a storage unit and a lifter unit. The storage unit has a storage frame, a storage rack and a first motor. The storage rack is adapted to store microscope slides. The storage rack is mounted to the storage frame and capable of sliding in a first plane by the first motor. The lifter unit has a loader frame, a loader arm and a second motor. The loader arm is mounted to the loader frame and capable of sliding in a second plane by the second motor. The loader arm is capable of accessing a selected microscope slide from the storage rack. The loader arm uses a vacuum chuck to retain the microscope slide on the loader arm. The loader arm may also have a motor to enable it to extend the vacuum chuck below the bottom surface of a microscope slide. After the microscope slide is secured to the loader arm, the microscope slide may be transported from the storage rack to a selected workstation such as an alignment mechanism. The retrieval unit is automated by having a computer control the operations of the motors. The present invention also includes a method within a microarray processing machine for retrieving a microscope slide in a storage rack and transporting the microscope slide to a workstation such as an alignment mechanism.

### CROSS REFERENCE TO RELATED PATENT APPLICATIONS

The present application claims priority from Provisional Application Serial No. ______ entitled “Microarray Retrieval Unit” filed Aug. 11, 2000.

This patent application is related to the following co-pending, commonly assigned patent applications, the disclosures of which are incorporated herein by reference in their entirety:

1. U.S. patent application Ser. No. ______, “Microarray Placer Unit,” by Bevirt, et al., filed concurrently herewith.

2. U.S. patent application Ser. No. ______, “Microarray Alignment Mechanism,” by Rollins, et al., filed concurrently herewith.

3. U.S. patent application Ser. No. ______ “Microarray Platen,” by Bevirt, et al., filed concurrently herewith.

4. Provisional Application Serial No. ______, “Microarray Placer Unit,” by Bevirt, et al., filed Aug. 11, 2000.

5. Provisional Application Serial No. ______, “Microarray Alignment Mechanism.” by Rollins, et al. filed Aug. 11, 2000.

6. Provisional Application Serial No. ______, “Microarray Platen,” by Bevirt, et al., filed Aug. 11, 2000.

### FIELD OF THE INVENTION

The present invention relates generally to a retrieval unit for the handling, transporting and processing of workpieces such as microscope slides and, more particularly, to a unit that retrieves workpieces before and after processing by a microarray processing machine or device.

### BACKGROUND OF THE INVENTION

A microarray processing machine or device processes DNA and other biological material for sampling and analysis. One type of microarray processing device is a microarray printer that deposits such genetic material on workpieces such as microscope slides. The microarray printer typically deposits the genetic material on microscope slides in a gridded array format. A high precision printing head is located on a robot arm that is movable on a gantry mechanism. The printing head is used to deposit thousands of small droplets of genetic material on the surface of the microscope slide.

Although microarray printers and other processing devices are commercially available, the current devices are not capable of automatically handling and transporting a large number of microarray slides before and after processing operations. Instead, prior to processing, current microarray printers require a user to physically place a microscope slide into position by hand on a worktable. The user may be required to align the edges of the microscope slides to certain reference points on the worktable. For a microarray printer, after the microscope slide is in position, the microarray printer is activated to deposit the DNA or other biological material needed for sampling and analysis. After processing by the microarray printer, a user physically removes the processed microarray slides from the worktable for subsequent analysis.

Several problems exist when a user is required to physically locate and remove microscope slides from a worktable. First, the possibility of error exists if the microscope slide is not aligned correctly. Second, and more importantly, the act of physically placing and removing microscope slides from a worktable is time consuming, especially if a user is processing a large number of microscope slides.

Thus, a need exists for automating the handling and transportation of microscope slides before and after processing by the microarray processing device. The present invention includes a microarray processing machine that overcomes the problems of conventional microarray processing devices To handle a large quantity of microscope slides before and after processing, an automated retrieval unit was developed to retrieve new microscope slides from a storage location. The retrieval unit transports the new microscope slides to an alignment mechanism for subsequent processing by the microarray processing device. After processing operations, the processed microscope slides are presented back to the alignment mechanism so that the microscope slides may be stored back to their original storage location.

### SUMMARY OF THE INVENTION

To that end, the present invention includes an automated microarray printer machine having the capability of transporting a plurality of microarray workpieces before or after printing operations by a printer device of the microarray printer machine. The automated microarray printer machine includes a storage unit, a workstation, and a retrieval mechanism. The storage unit is used to store the plurality of microarray workpieces. The retrieval mechanism is used to transport one of the microarray workpieces from the storage unit to the workstation such as an alignment mechanism.

In another embodiment, the present invention also provides a retrieval unit for a microarray processing device to transport a workpiece such as a microscope slide before or after processing operations by the microarray processing device. The retrieval unit includes a storage unit and a lifter unit. The storage unit has a storage frame, a storage rack and a first motor. The storage rack is adapted to store microscope slides. The storage rack is mounted to the storage frame and capable of sliding in a first plane by the first motor. The lifter unit has a loader frame, a loader arm and a second motor. The loader arm is mounted to the loader frame and capable of sliding in a second plane by the second motor. The loader arm is capable of accessing a selected microscope slide from the storage rack. The loader arm uses a vacuum chuck to retain the microscope slide on the loader arm. The loader arm may also have a motor to enable it to extend the vacuum chuck below the bottom surface of a microscope slide. After the microscope slide is secured to the loader arm, the microscope slide may be transported from the storage rack to a selected workstation such as an alignment mechanism. The retrieval unit is automated by having a computer control the operations of the motors.

The storage rack may also include a plurality of slots adapted to receive a plurality of microscope slide containers. Each microscope slide container would be capable of retaining a plurality of microscope slides. Each slot may further include an optical interrupter sensor or pressure sensor to determine whether a microscope slide container is present in the slot. The sensor may also determine whether the container is properly secured in the slot.

In another embodiment, the present invention is an automated microarray printer for performing printing operations on microscope slides. The automated microarray printer has a computer to control the transportation of microscope slides before or after the printing operations. Such a microarray printer may include a retrieval unit capable of retrieving microscope slides from a storage area. In particular, the retrieval unit has a storage unit and a lifter unit. The storage unit has a storage frame, a storage rack and a first motor. The first motor is controlled by the computer and adapted to slide the storage rack in a first plane. The lifter unit has a loader frame, a loader arm and a second motor. The second motor is also controlled by the computer and adapted to slide the loader arm in a second plane. The loader arm also has a vacuum chuck to hold a selected microscope slide in position on the loader arm.

In a further embodiment, the present invention is a method within a microarray printer for retrieving a microscope slide in a storage rack and transporting the microscope slide to a Workstation such as an alignment mechanism. The microarray printer has a loader arm with a vacuum chuck. The method includes the steps of: determining the location of the microscope slide in the storage rack; moving the loader arm in close proximity to the selected microscope slide; extending the vacuum chuck under the microscope slide; activating the vacuum chuck to hold the microscope slide on the loader arm; and moving the loader arm and microscope slide to the workstation.

The determining step may include receiving a set of coordinates for the selected microscope slide from a computer. The step of moving the loader arm in close proximity may include activating at least one motor that slides either the loader arm or the storage rack in a vertical or horizontal plane. The extending step may include activating another motor on the loader arm that slides the vacuum chuck underneath the selected microscope slide. Activating the vacuum chuck may include opening a valve to expose the vacuum chuck to a vacuum source. The step of moving the loader arm to the workstation may include activating at least one motor that slides the loader arm to the workstation.

In yet another embodiment, the present invention is a method within a microarray printer for retrieving a microscope slide in a storage rack and transporting the microscope slide to a workstation such as an alignment mechanism. The microarray printer has a first motor to slide the storage rack in a first plane. The microarray printer also has a second motor to slide a loader arm in a second plane. The loader arm has a vacuum chuck for holding the microscope slide in position. In this embodiment, the method includes the steps of: determining the location of the microscope slide in the storage rack; moving the loader arm in close proximity to the selected microscope slide by activating the first and second motors; extending the vacuum chuck toward or under the microscope slide; activating the vacuum chuck to hold the microscope slide on the loader arm; and moving the loader arm and microscope slide to the workstation by activating the second motor.

In a further embodiment, the present invention is a retrieval unit for a microarray processing device to transport a workpiece before or after processing operations by the microarray processing device. The retrieval unit includes a storage unit and a lifter unit. The storage unit has a storage frame, a storage rack and a first means for translating the storage rack in a first plane. The lifter unit has a loader fram, a loader arm and a second means for translating the loader arm in a second plane. The loader arm is capable of accessing and retrieving a workpiece stored in the storage rack.

The above summary of the present invention is not intended to represent each embodiment, or every aspect of the present invention. This is the purpose of the figures and detailed description that follows.

### BRIEF DESCRIPTION OF THE DRAWINGS

Other objects and advantages of the invention will become apparent upon reading the following detailed description and upon reference to the drawings.

FIG. 1 is a perspective view of a microarray printer containing embodiments of the present invention.

FIGS. 2A and 2B are top level block diagrams illustrating the general transport flow of a microscope slide before and after printing operations.

FIGS. 3A and 3B are perspective views of one embodiment of a retrieval unit of the present invention with and without protective covers.

FIG. 3C is a side view of a retrieval unit of the present invention.

FIG. 4 is a perspective view of a storage rack of the retrieval unit in FIGS. 3A-3C.

FIG. 5A is a perspective view of one embodiment of a lifter unit of the retrieval unit in FIGS. 3A-3C.

FIGS. 5B and 5C are perspective views of one embodiment of a loader arm for the lifter unit in FIG. 5A.

FIGS. 6A-6C illustrates how the retrieval unit in FIGS. 3A-3B operate;

FIG. 6A shows a loader arm of the lifter unit as it accesses a microscope slide from the storage unit.

FIG. 6B shows a loader arm of the lifter unit as it removes a microscope slide from the storage unit.

FIG. 6C shows a loader arm with a microscope slide traversed to the top of the lifter unit for presenting the slide to the next workstation such as an alignment mechanism.

FIG. 7 is a flow diagram showing the steps in one embodiment for automatically retrieving a microscope slide from the storage unit to the alignment mechanism.

FIG. 8 is a flow diagram showing the steps in one embodiment for automatically retrieving a microscope slide from the alignment mechanism and returning the microscope slide to the storage unit.

FIG. 9 is a perspective view of one embodiment of a microarray alignment mechanism of the present invention.

FIG. 10 is a top view of the microarray alignment mechanism in FIG. 9.

FIG. 11A is a perspective view of the top of the microarray alignment mechanism in FIG. 9 without its housing.

FIG. 11B is a perspective view of the bottom of the microarray alignment mechanism in FIG. 9 without its housing.

FIGS. 12A-12D are bottom views of various stages in the assembly process of the microarray alignment mechanism in FIG. 9.

FIG. 13 is a bottom view the microarray alignment mechanism in FIG. 9 without its housing.

FIGS. 14A-14D are various top views of the microarray alignment mechanism in FIG. 9 as it captures and takes possession of a microscope slide from the retrieval unit.

FIG. 15 is a perspective view of one embodiment of a printer device and placer unit of the present invention mounted on a movable gantry.

FIG. 16 is a top view of the placer unit and movable gantry of FIG. 15.

FIG. 17 is a perspective view of the printer device and placer unit of FIG. 15.

FIG. 18 is a perspective view of one embodiment of a placer unit mounted on a printer carriage.

FIG. 19 is a side view of one embodiment of a placer unit mounted on a printer carriage.

FIGS. 20A and 20B are perspective views of one embodiment of a placer unit.

FIG. 21 is a perspective view of one embodiment of a platen of the present invention.

FIG. 22 is a closer view of a section of the top surface of the platen illustrated in FIG. 21.

FIG. 23 is a plumbing diagram of one embodiment of a vacuum assembly used in connection with the platen in FIG. 21.

FIGS. 24A and 24B are perspective and top views of the valve and pressure sensor assembly for the platen in FIG. 21.

FIGS. 24C and 24D are sectional views of the valve and pressure sensor assembly for the platen in FIG. 21.

FIG. 25 is a closer view of a section of the bottom surface of the platen illustrated in FIG. 21.

FIG. 26A is a perspective view of one embodiment of a positioning device of the present invention.

FIG. 26B is a sectional view of the positioning device shown in FIG. 26A.

While the invention is susceptible to various modifications and alternative forms, certain specific embodiments thereof have been shown by way of example in the drawings and will be described in detail. It should be understood, however, that the intention is not to limit the invention to the particular forms described. On the contrary, the intention is to cover all modifications, equivalents, and alternatives falling within the spirit and scope of the invention as defined by the appended claims.

### DESCRIPTION OF ILLUSTRATIVE EMBODIMENTS

Illustrative embodiments will now be described with reference to the accompanying figures. The invention relates to an apparatus and method to automate the handling and transporting of microscope slides before and after main processing operations by a microarray processing device. The invention reduces the amount of human intervention while increasing throughput. Although the specific embodiment used to describe the present invention is an automated microarray printer, it is contemplated that the present invention may be applicable to other types of microarray processing machines or devices. Further aspects and advantages of the invention will become apparent from consideration of the following description and drawings.

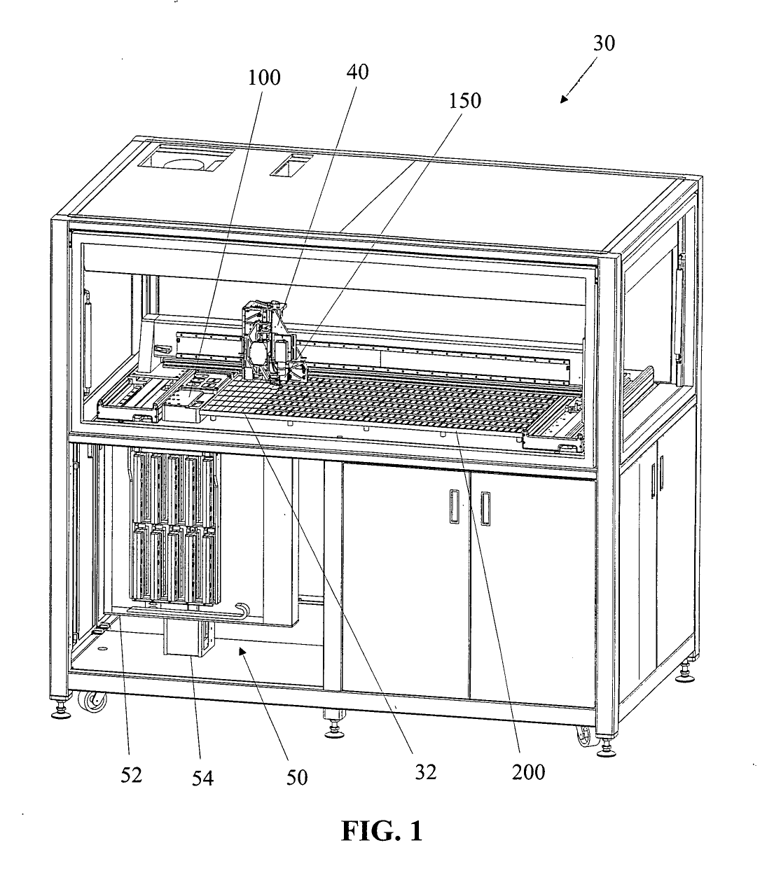

Turning to the drawings, FIG. 1 depicts a microarray printer 30 containing embodiments of the present invention. The microarray printer 30 of the present invention generally includes a printer device 40, a retrieval unit 50, an alignment mechanism 100, a placer unit 150, and a worktable or platen 200. The printer device 40 of the microarray printer 30 deposits DNA or other biological material on workpieces or microscope slides 32 for sampling and analysis. The microarray printer 30 performs its main printing or processing operations when the microscope slides 32 are located on the platen 200. The microarray printer 30 is automated and can handle a large number of microscope slides 32 without a user having to physically place microscope slides 32 on the platen 200. In particular, the microarray printer 30 uses the retrieval unit 50, the alignment mechanism 100, and the placer unit 150 to handle and transport microscope slides 32 before and after its main processing operations.

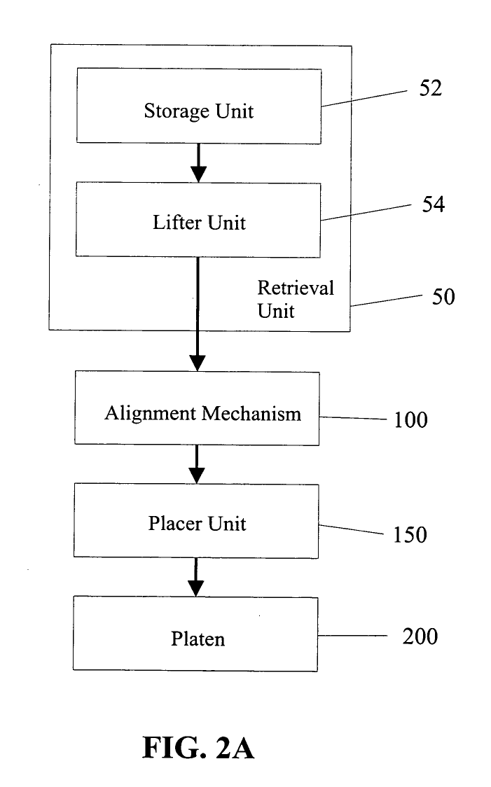

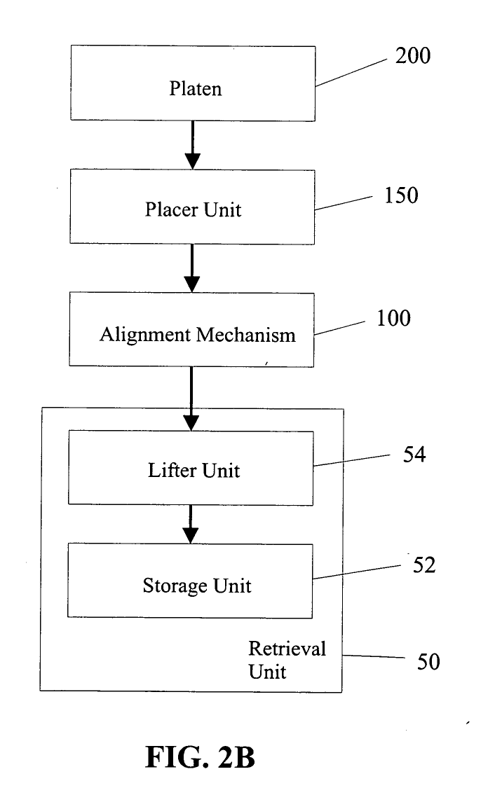

Although specific details on the handling and transporting of microscope slides 32 are described below, FIG. 2A is a diagram describing the general flow of a new microscope slide 32 before printing operations. A top level description of the general transport flow follows. The retrieval unit 50 has two main components: the storage unit 52 and the lifter unit 54. New microscope slides 32 are initially stored in the storage unit 52 of the retrieval unit 50. When processing a new microscope slide 32, the lifter unit 54 retrieves the new microscope slide 32 and presents the slide 32 to the alignment mechanism 100. The alignment mechanism 100 captures and takes possession of the new microscope slide 32. The alignment mechanism 100 precisely positions the new microscope slide 32 and presents the slide 32 to the placer unit 150. The placer unit 150 then picks up the new microscope slide 32 and positions the slide 32 on the platen 200. After the new microscope slide 32 is secure on the platen 200 the printer device 40 may deposit the genetic material on the new microscope slide 32. The retrieval unit 50, alignment mechanism 100, and placer unit 150 may operate asynchronously with respect to each other. In other words, the retrieval unit 150 array be locating or transporting a new microscope slide 32 at the same time the placer unit 150 is transporting or positioning another microscope slide 32 on the platen 200.

FIG. 2B is a diagram describing the general flow of a processed microscope slide 32 after printing operations. Although a more detailed description is provided later, a top level description of the general transport flow of a processed microscope slide 32 follows. The placer unit 150 picks up the processed microscope slide 32 from the platen 200 and transports the slide 32 to the alignment mechanism 100. The alignment mechanism 100 then captures and takes possession of the processed microscope slide 32. The lifter unit 54 of the retrieval unit 50 then slides into position underneath the processed microscope slide 32. The lifter unit 54 grabs the processed microscope slide 32 and then transports the slide 32 to the storage unit 52 where it may be removed by a user for subsequent analysis. Again, the retrieval unit 50 alignment mechanism 100, and placer unit 150 may operate asynchronously with respect to each other.

Detailed descriptions of the retrieval unit 50, the alignment mechanism 100 the placer unit 150, and the platen 200 follows.

Retrieval Unit

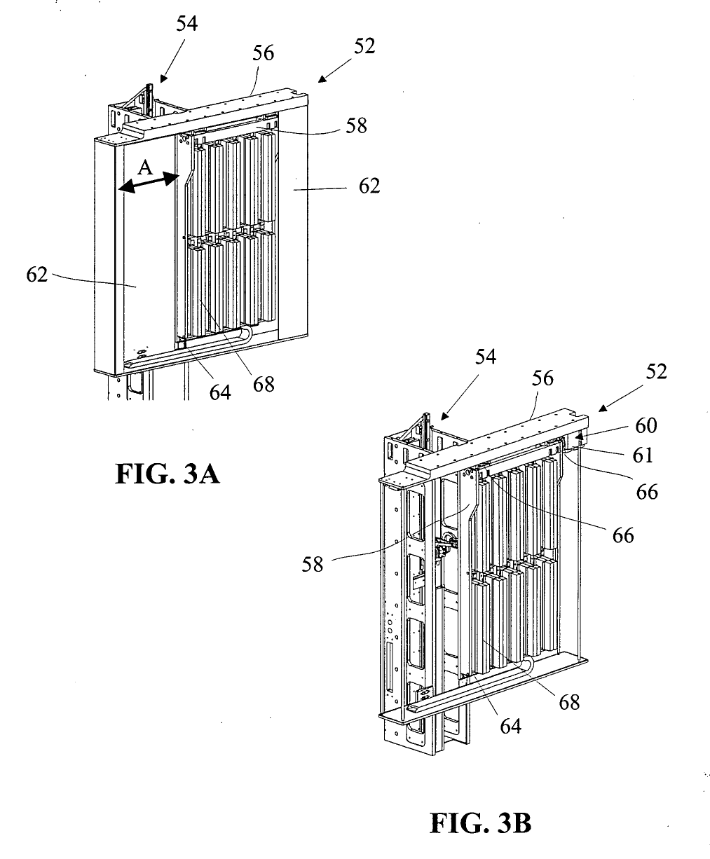

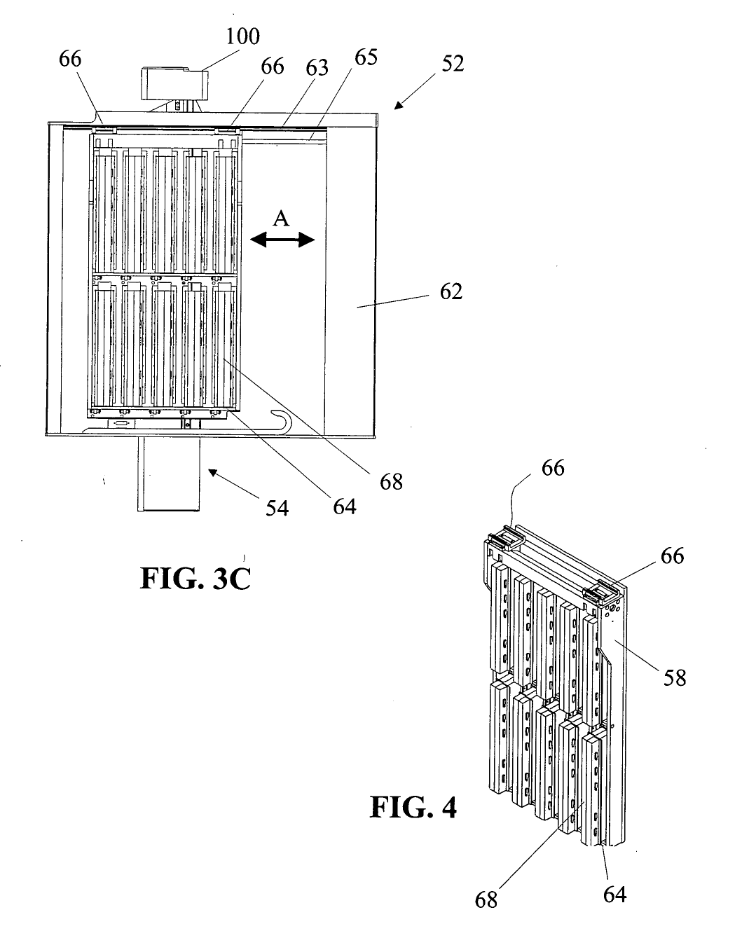

Referring to FIGS. 3A-3C, the retrieval unit 50 includes two main components, a storage unit 52 and a lifter unit 54. One purpose of the retrieval unit 50 is to access new microscope slides 32 stored in the storage unit 52, remove the new microscope slides 32 from the storage unit 52 and present the new microscope slides 32 to another workstation such as the alignment mechanism 100 for further handling by the microarray printer 30. Another purpose of the retrieval unit 50 is to remove processed microarray slides 32 from a workstation such as the alignment mechanism 100 and replace processed microarray slides 32 back into the storage unit 52.

The storage unit 52 includes a storage frame 56, a storage rack 58, and a rack motor assembly 60. The storage frame 56 is rigidly attached to the microarray printer 30. The storage frame may also include protective covers 62. The storage rack 58 is mounted on a linear rail 64 on the storage frame 56 and is capable of traversing in the horizontal direction as illustrated by arrow A in FIGS. 3A and 3C. As shown in FIG. 3, the storage rack is clamped to the linear rail on the storage frame 56 via rail mounts 66. The rack motor assembly 60 enables the storage rack 58 to traverse along the linear rail of the storage frame 56. The rack motor assembly 60 may be electrically controlled by a computer system. The rack motor assembly 60 is rigidly mounted to storage frame 56 or the storage rack 58. The rack motor assembly 60 may include a brushless DC motor 61 that drives on a lead screw 65 to move the storage rack 58 in the horizontal direction. It is contemplated that the rack motor assembly may translate by other means such as a pneumatic actuator that slides the storage rack 58 in the horizontal direction. As described in more detail below, the horizontal movement of the storage rack 58 enables the lifter unit 58 to access microscope slides 32 in the horizontal direction.

Referring to FIGS. 3A-3C and 4, in one embodiment, the storage rack 56 has a plurality of slots 64 to receive and hold a plurality of microscope slide containers 68. Each slot 64 may further include a sensor to enable a monitoring computer system to determine whether a microscope slide container 68 is properly secured within a slot 64 of the storage rack 56. The sensor is preferably an optical interrupter sensor but may also be another type of sensor such as a pressure sensor. Each microscope slide container 68 is capable of retaining a plurality of microscope slides 32. The use of microscope slide containers 68 enables a user to easily remove and replace large quantities of microscope slides 32. Accordingly, the protective covers 62 should not block exterior access to the microscope slide containers 68. An exemplary design of a microscope slide container is disclosed in provisional application Serial Number 60/211,233, entitled “Microscope Slide Container” and filed on Jun. 12, 2000, which is owned by the assignee of the present application and incorporated herein by reference in its entirety.

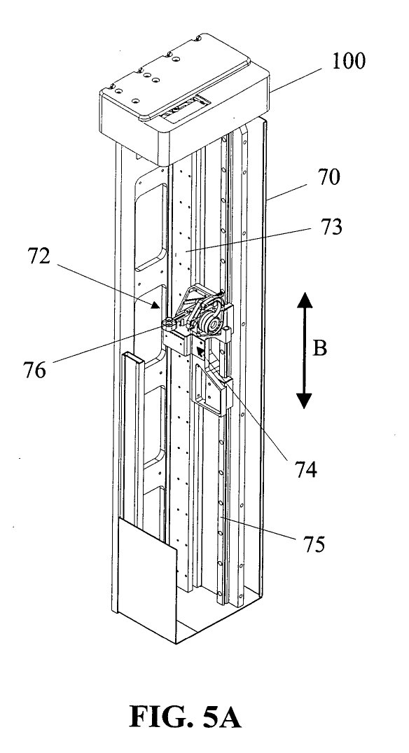

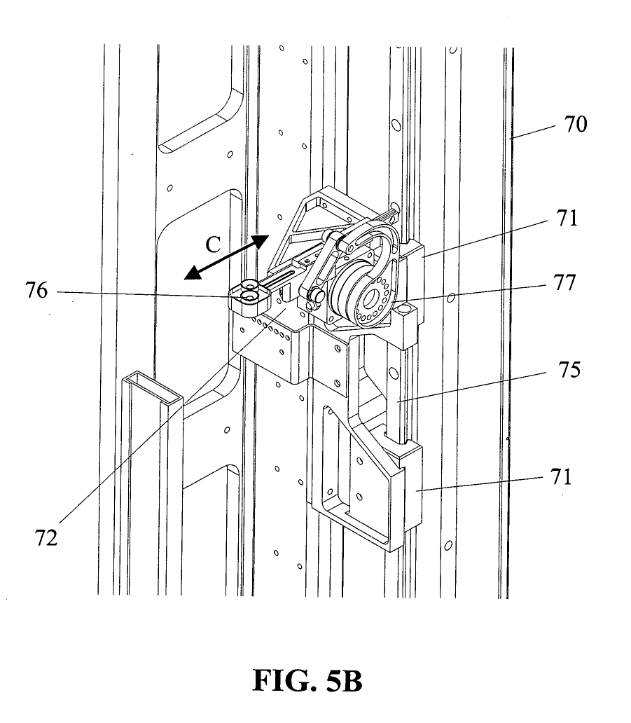

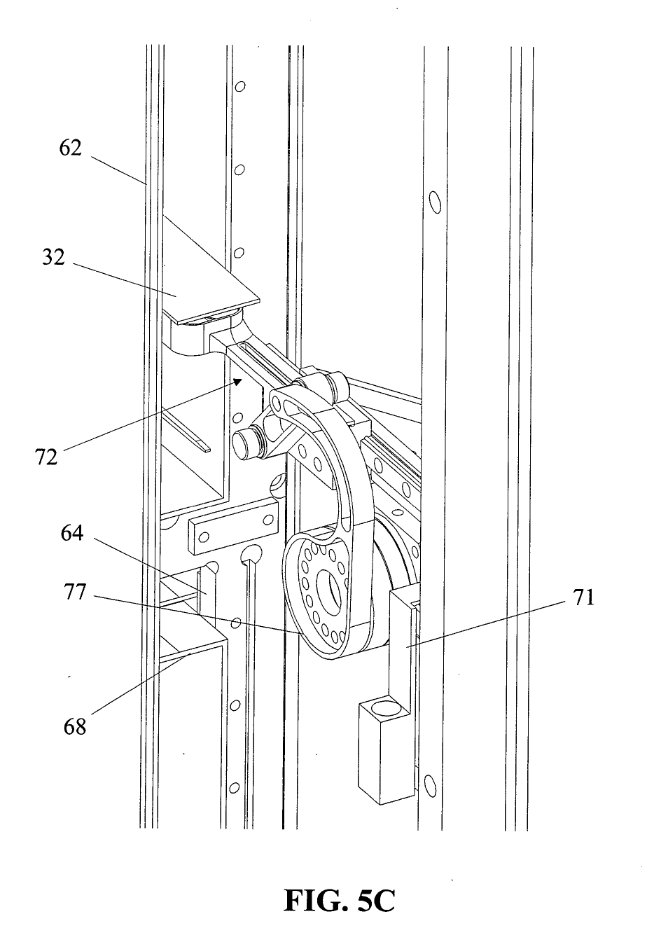

Referring to FIG. 5A, the lifter unit 54 includes a loader frame 70 a loader arm 72, and a lifter motor assembly 74. The loader frame 70 is rigidly attached to the microarray printer 30 and provides the structural support for the loader arm 72 to traverse in the direction as shown by arrow B. The loader frame 70 has a linear rail 75 to guide the loader arm 72 in direction or plane B. The loader arm 72 is attached to the linear rail 75 via linear bearings 71. In one embodiment, the lifter motor assembly 74 includes a linear motor 73 which is rigidly attached to the loader frame 70. In another embodiment, the lifter motor assembly 74 may include a brushless DC motor that drives a lead screw to permit the loader arm 72 to move up and down. In further embodiments, a pneumatic actuator may be used to slide the loader arm 72 in the vertical direction instead of the lifter motor assembly 74.

Referring to FIG. 5B, the loader arm 72 has at least one suction cup or vacuum chuck 76 to secure microscope slides 32 to the loader arm 72. The vacuum chuck 76 may be connected to a vacuum source via a flexible hose. The loader arm 72 further includes a loader arm motor 77 that enables the loader arm 72 to traverse in the direction as shown by arrow C. Movement of the loader arm 72 in the direction or plane C allows the loader arm to remove and replace microscope slides 32 from the microscope slide containers 68. FIG. 5C shows the loader arm 72 in the extended position as it retrieves a microscope slide 32 from the storage unit 52. In the view as shown in FIG. 5C, the loader arm motor 77 rotates in the counter-clockwise direction to extend the vacuum chuck 76. The loader arm motor 77 rotates in the clockwise direction to retract the vacuum chuck 76.

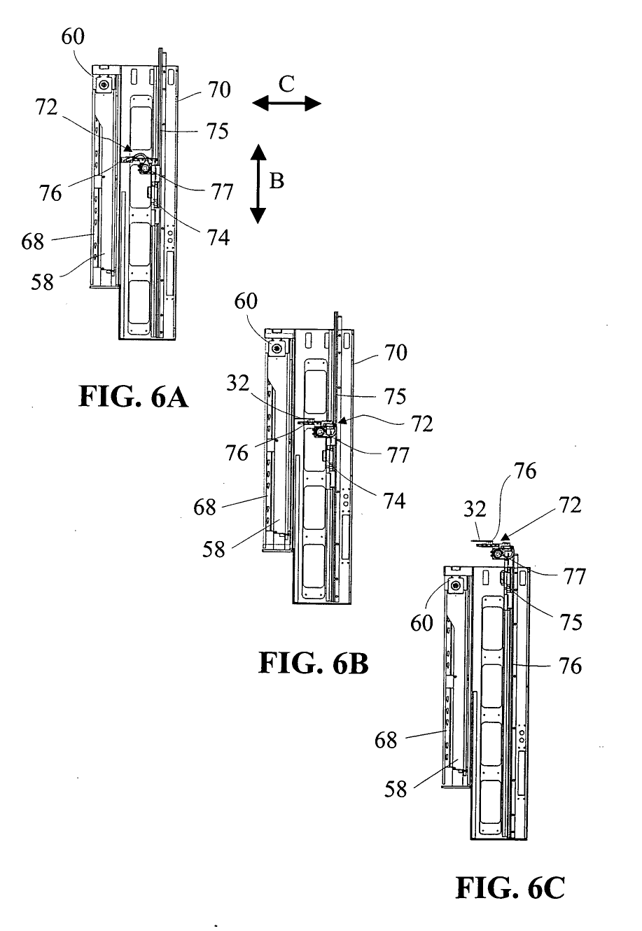

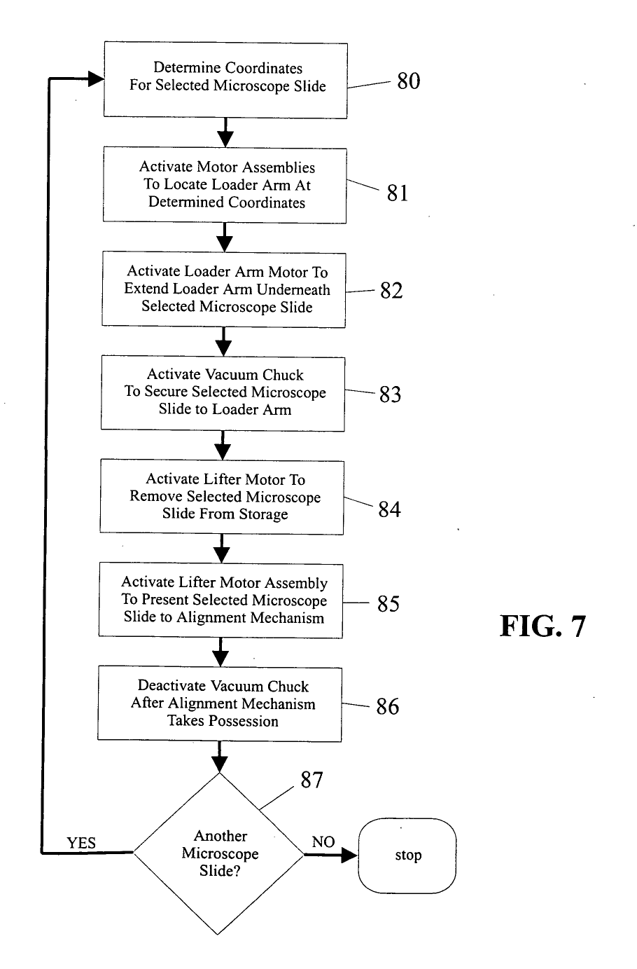

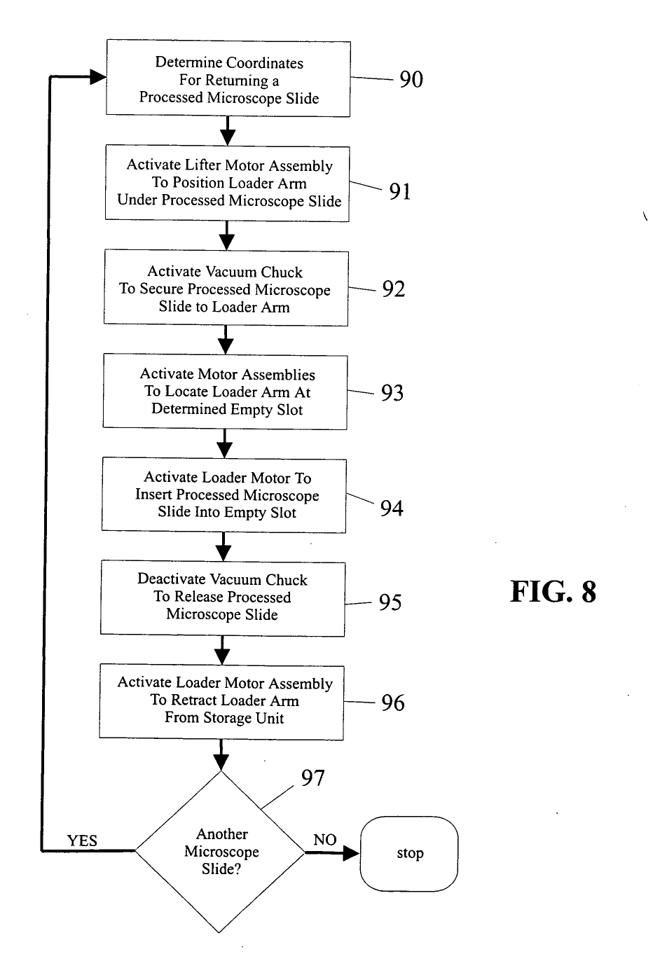

The steps for handling microscope slides 32 within the retrieval unit are automated by a computer with the use of a program. The program controls the steps outlined below and illustrated in FIGS. 6A-6C and the flow diagrams of FIGS. 7 and 8. The present invention is not limited to the number of steps or order of steps outlined in FIGS. 7 and 8. A person skilled in the art, having the benefit of this specification, may realize additional steps or a different sequence of steps.

FIG. 7 illustrates the steps that may be taken in one embodiment for retrieving a selected microscope slide 32 from the storage unit 52 and presenting the selected microscope slide 32 to the alignment mechanism 100. In step 80, the program determines or receives information such as coordinates for the selected microscope slide 32. The coordinates may be pre-programmed so the slides in several containers 68 may be processed. Alternatively, it is contemplated that the information on the desired location of the slide may be separately entered by the user. In step 81, the program will activate the lifter motor assembly 74 to traverse the loader arm 72 in the vertical direction B toward the selected microscope slide 32. During this step, the program also activates the rack motor assembly 60 so that the storage rack 58 traverses in the horizontal direction A to further locate a specific microscope slide 32. The program uses the determined or received information or coordinates to locate the selected microscope slide 32. In step 82, and illustrated in FIG. 6A, the program activates the loader arm motor 77 so that the loader arm 72 and vacuum chuck 76 extends toward the selected microscope slide 32 in the direction C to engage the bottom surface of the selected microscope slide 32. In step 83, after the loader arm 72 and vacuum chuck 76 are positioned under the bottom surface of the selected microscope slide 32, the program activates the vacuum chuck 76 of the loader arm 72. This secures the microscope slide 32 to the loader arm 72. As will become apparent below, the microscope slide 32 is preferably handled on the bottom side of the microscope slide 32 so that the sides and corners of the microscope slide 32 may be aligned later by the alignment mechanism 100. Moreover, the handling of the bottom of the microscope slide 32 will avoid contamination if the loader arm 72 is handling the transportation of processed microscope slides 32. In step 84, as illustrated in FIG. 6B, the program activates the loader arm motor 77 so that the loader arm 72 removes the microscope slide 32 from the microscope slide container 68. The motor 77 traverses the loader arm 72 in the horizontal direction C. In step 85, as illustrated in FIG. 6C, the program activates the lifter motor assembly 74 to traverse the loader arm 72 in the upward direction. The loader arm 72 lifts the selected microscope slide 32 to the top of the lifter unit to present the microscope slide 32 to the next workstation such as the microarray alignment mechanism 100. In step 86, the program deactivates the vacuum chuck 76 so that the alignment mechanism 100 may take possession of the microscope slide 32. After passing the microscope slide 32 to the alignment mechanism 100, the program will determine in step 87 whether additional microscope slides 32 need to be transported to the alignment mechanism 100. If so, the program will repeat the above steps.

As described in FIG. 8, a similar approach may be taken when a processed microscope slide 32 located in the microarray alignment mechanism 100 needs to be transported back to the storage unit 52. In step 90, the program determines or receives information such as coordinates on where the processed microscope slide 32 needs to be stored. In one embodiment, the program keeps track of filled and empty slots in the microscope slide containers 68 and may check whether a particular slot is empty. In step 91, the program activates the lifter motor assembly to traverse the loader arm 72 in the vertical direction B to the top of the lifter unit 54. In step 92, after the loader arm 72 is positioned under the bottom surface of the processed microscope slide 32, the vacuum chuck 76 of the loader arm 72 is activated. This secures the microscope slide 32 to the loader arm 72. The alignment mechanism 100 may then release the processed microscope slide 32. In step 93, the program then activates the lifter motor assembly 74 to traverse the loader arm 72 with the processed microscope slide 32 in the vertical direction B toward the empty microscope slide slot. During this step, the program also activates the rack motor assembly 60 so that the storage rack 58 traverses in the horizontal direction A to further locate a specific empty slot. The program uses the determined or received information such as coordinates to locate the empty slot. In step 94, the program activates the loader arm motor 77 so that the loader arm 72 extends the processed microscope slide 32 into the empty slot. In step 95, after the loader arm 72 is positioned in the slot, the program deactivates the vacuum chuck 76 of the loader arm 72. This releases the processed microscope slide 32 from the loader arm 72. In step 96, the program activates the loader arm motor 77 so that the loader arm 72 retracts from the processed microscope slide 32. After placing the processed microscope slide 32 into the empty slot, the program will determine in step 97 whether additional microscope slides 32 need to bc transported to empty slots in the storage unit 52. If so, the program will repeat the above steps. Alternatively, the program may determine whether new microscope slides 32 need to be transported from the storage unit 52 to the alignment mechanism 100 for processing by the printer.

It is noted that the geometric design of the loader arm 72 may provide limits on which microscope slide 32 may be obtained or returned to the storage unit 52. For example, if the microscope slides 32 are closely stored or packed in a microscope slide container 68, the loader arm 72 may only be able to remove or replace a microscope slide 32 in the lowest available position in the microscope slide container 68. Thus, the design of loader arm 72 and the microscope slide container 68 may need to be considered when implementing the above retrieval and replacement operations. For instance, if the microscope slides 32 are closely stored in the microscope slide container 68, then the computer system should be designed to retrieve a microscope slide 32 from the lowest position in a container 68. Similarly, where the microscope slides 32 are closely stored in the microscope slide container 68, the microscope slide 32 should be first returned to the highest available position in a container 68.

Alignment Mechanism

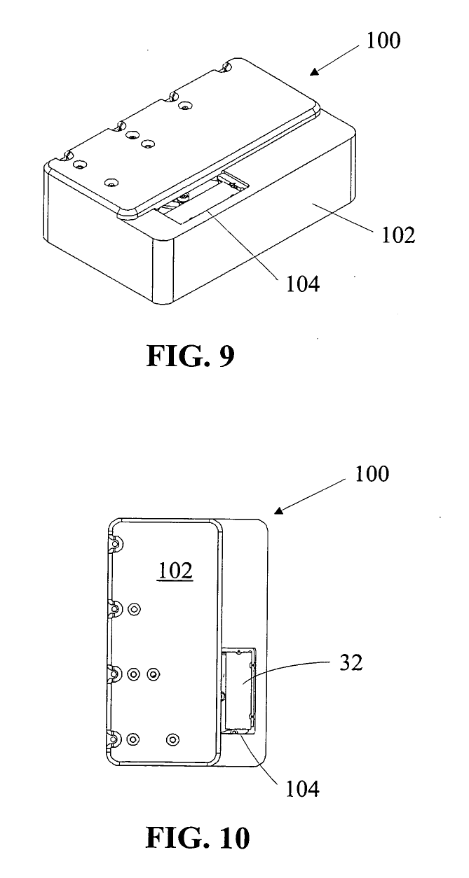

FIGS. 9 and 10 illustrate one embodiment of the alignment mechanism 100 of the present invention with its housing 102. For purposes of the system depicted in FIG. 1, the main purpose of the alignment mechanism 100 is to receive new microscope slides 32 from the retrieval unit 50 and align the slide 32 so that it may be transported to the platen 200 by the placer unit 150. An aligned microscope slide 32 is presented to the placer unit 150 via opening 104 in the housing 102. Another purpose of the alignment mechanism 100 is to receive processed microscope slides 32 from the placer unit 150 and align the slide 32 so that it may be transported to the storage unit 52 by the retrieval unit 50. Processed microscope slides 32 are received from the placer unit 150 via opening 104 in the housing 102. The alignment mechanism 100 aligns the microscope slides 32 so that the system can register and know the exact position of a microscope slide 32. In other words, the alignment mechanism 100 converts a randomly-oriented microscope slide 32 contained within a certain service envelope to a precisely-positioned microscope slide 32 in three axes.

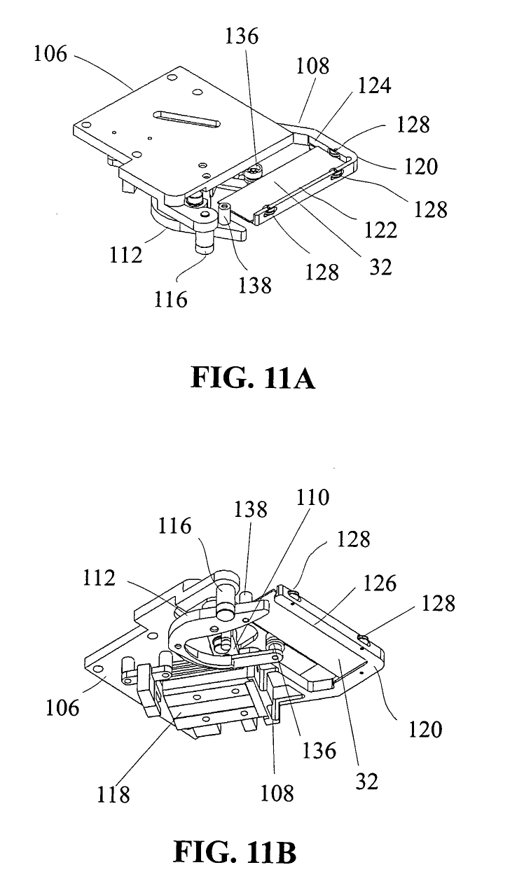

FIGS. 11A and 11B are perspective views of the top and bottom of one embodiment of the alignment mechanism 100 without housing 102. The main components of the alignment mechanism 100 include a stationary support base 106, a actuator arm 108, a pivotal member 110 and a roller support member 112. The movable parts of the alignment mechanism include the actuator arm 108, the pivotal member 110 and the roller support member 112.

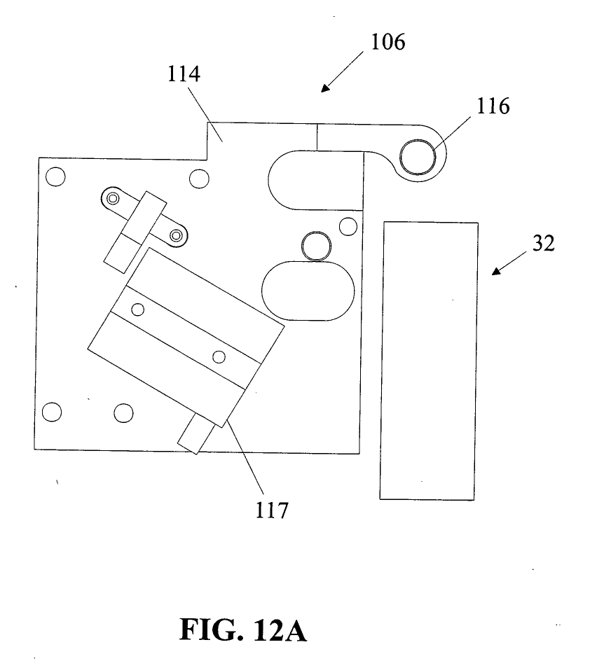

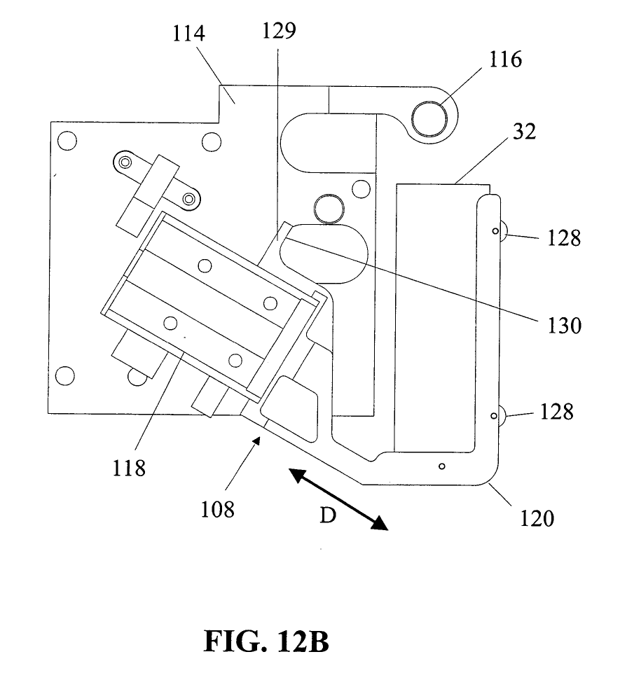

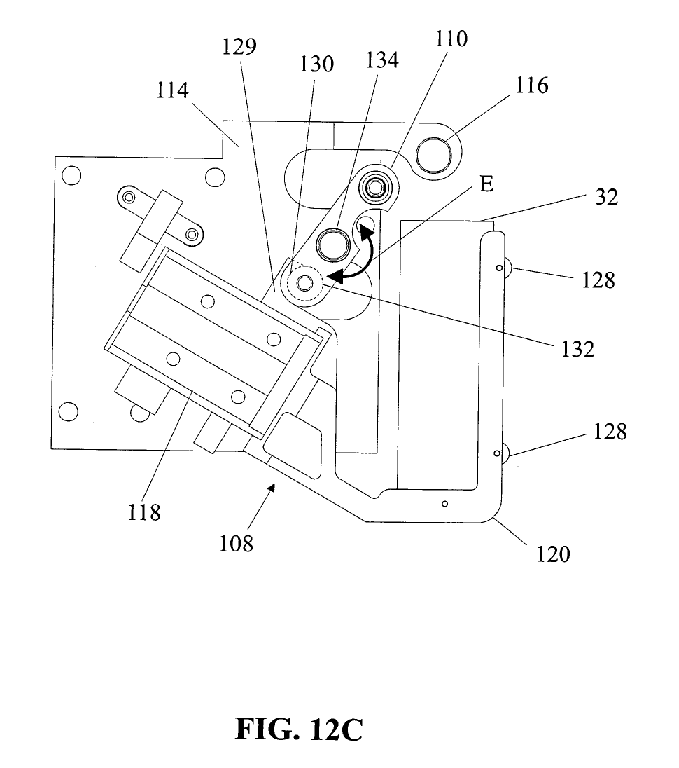

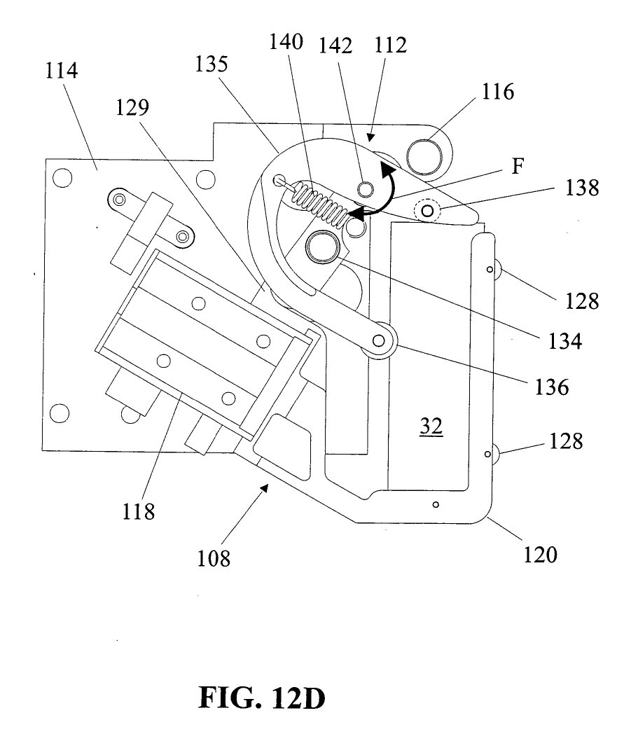

FIGS. 12A-12D are various bottom views of alignment mechanism 100 during assembly. Microscope slide 32 is shown as a reference. These views will assist in explaining the operation of one embodiment of the present invention. FIG. 12A illustrates the stationary support base 106. The stationary support base 106 is rigidly attached to the housing 102. The stationary support base 106 includes a support plate 114, a roller 116 and an actuator mount rail 117.

FIG. 12B illustrates the stationary support base 106 and the actuator arm 108. The actuator arm 108 is attached slidably to the bottom side of the support plate 114 via the actuator mount rail 117. The actuator arm 108 includes an actuator 118 and a registration arm 120. In the preferred embodiment, the actuator 118 is a guided pneumatic actuator although the present invention is not limited to the use of a pneumatic actuator. It is contemplated that the actuator 118 could be replaced with an electrical actuator or a motor. The registration arm 120 is rigidly attached to the actuator 118. The actuator 118 is capable of sliding in direction D as show n in FIG. 12B. The registration arm 120 has a first registration surface 122, a second registration surface 124, a third registration surface 126, and springs 128 as shown in FIGS. 11A and 11B. The actuator arm 108 has an extended member 129 that defines a roller contact surface 130.

FIG. 12C illustrates the stationary support base 106, the actuator arm 108, and the pivotal member 110. The pivotal member has a roller 132. When actuator 118 moves in direction D, the roller contact surface 130 makes contact with and presses against roller 132. The pressing of the roller 132 causes member 110 to pivot about a pivot joint 134 in direction as shown by arrow E in FIG. 12C. The pivot joint 134 is attached to the stationary support base 106.

FIG. 12D illustrates the assembly of the stationary support base 106, the actuator arm 108, the pivotal member 110, and the roller support member 112. The roller support member 112 has a main body portion 135 a first roller 136 and a second roller 138. In one embodiment, the main body portion 135 is substantially U-shaped. The roller support member 112 is attached to the stationary support base 106 by tension spring 140. The roller support member 112 is also slidably attached to the stationary support base 106 via pivotal member 110. The roller support member 111 is pivotally attached to the pivotal member 110 at pivot joint 142. As mentioned above the pivotal member 110 is pivotally attached to the stationary support base 106 at pivot joint 134. The roller support member 110 is allowed to pivot about pivot joint 142 in direction as shown by arrow F in FIG. 12D.

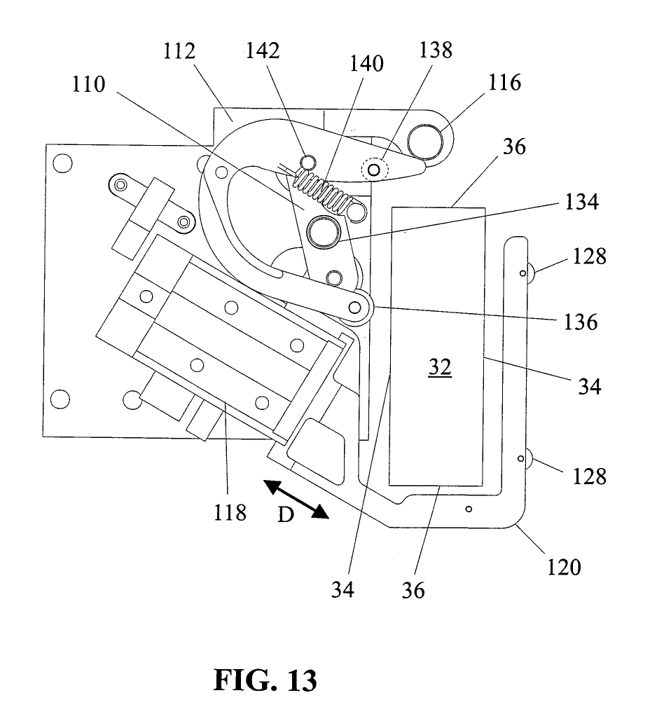

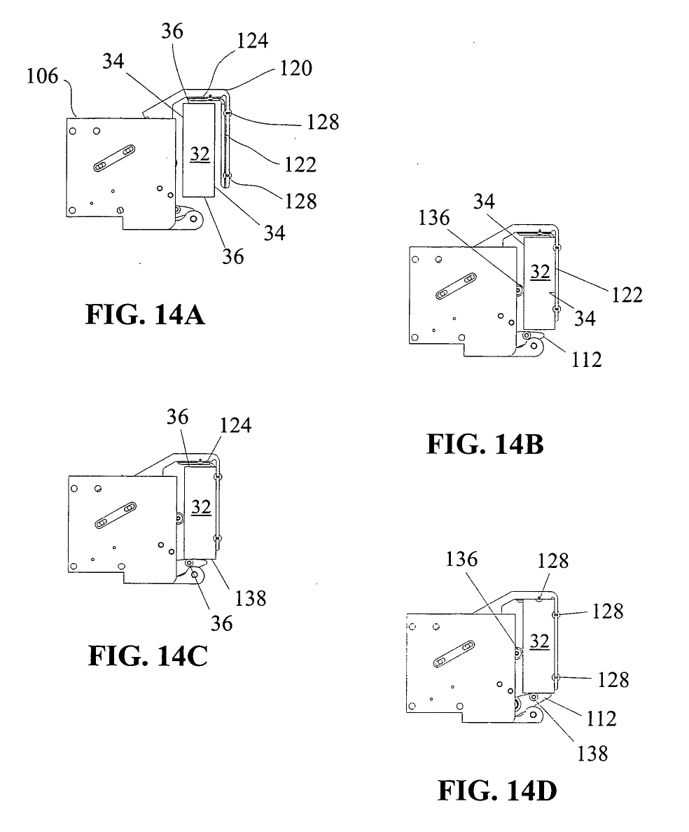

The steps for aligning a microscope slide 32 within the alignment mechanism 100 are automated by activating the actuator 118. The movement of the actuator 118 may controlled by a computer. It is preferable to use a single actuator for performing the alignment operations. A single actuator avoids having to precisely choreograph multiple actuators. The alignment of a microscope slide 32 is explained with reference to FIGS. 13 and 14A-14D.

FIGS. 13 and 14A illustrate a microscope slide 32 that has been presented to the alignment mechanism 100 within its working envelope. For purposes of describing the alignment operations, references to the long edges 34 and short edges 36 of the microscope slide 32 will be made. Before alignment, the actuator 118 is placed in a position such that the registration arm 120 is in the farthest location from the microscope slide 32. At this initial point, as shown in FIG. 13, the roller contact surface 130 of the actuator 118 is also pressing against roller 132 and pivoting joints 134 and 142 such that the roller support member 112 is also located in the farthest location from the microscope slide 32. This is illustrated in FIGS. 13 and 14A.

After the microscope slide 32 is within its working envelope, the actuator 118 is activated to translate or slide the registration arm 120 toward the microscope slide 32 in direction D. Activating the actuator 118 will also release pressure of the roller contact surface 130 against roller 132 of the pivotal member 110. This causes the pivotal member 110 to pivot about pivot joint 134 in a clockwise direction (if viewing the pivot joint 134 from the bottom view in FIG. 13). This in turn causes the tension spring 140 to pivot the roller support member 112 toward the microscope slide 32. As shown in FIG. 14B, the U-shape design of the roller support member 112 enables the first roller 136 to make contact with one of the long edges 34 of the microscope slide 32. The first roller 136 presses the other long edge 34 of the microscope slide 3 against the first registration surface 122 of the registration arm 120. This secures the long edge 34 of the microscope slide 32. The roller support member 112 continues to pivot toward the microscope slide 32. As illustrated in FIG. 14C, the second roller 138 then presses one of the short edges 36 of the microscope slide 32 against the second registration surface 124 of the registration arm 120. This secures the short edges 36 of the microscope slide 32. By pressing the long edge 34 of the microscope slide 32 before the short edge 36, the risk of skewing the microscope slide 32 during alignment is reduced. As the roller support member 112 pivots harder toward the microscope slide 32, the springs 128 press against the top surface of the microscope slide 32 as shown in FIG. 14D. The springs 128 force the bottom surface of the microscope slide 32 to press against the third registration surface 126 of the registration arm 120. The springs 128 are preferably in the shape of angled discs so that the microscope slide 32 becomes wedged between the springs 128 and the third registration surface 126. The alignment of the microscope slide 32 is now complete.

It is noted that the use of the tension spring 140 provides the added benefit of protecting the microscope slide 32 when fluctuations of air pressure exist in the pneumatic air supply to the actuator 118 if the actuator 118 is a pneumatic actuator. Excessive air pressure may translate to excessive pressure by the registration arm 120 when grabbing the microscope slide 32. The tension spring 140 enables a more constant pressure on the edges of the microscope slide 32 despite fluctuations in air pressure.

After aligning the microscope slide 32, it may be necessary to release the microscope slide 32 so that the slide 32 may be moved to another workstation. To release the microscope slide 32, the actuator 118 is activated to translate or slide the registration arm 120 away from the microscope slide 32 in direction D. Activating the actuator 118 will also push the roller contact surface 130 against the roller 132 of the pivotal member 110. This causes the pivotal member 110 to pivot about pivot joint 134 in the counter-clockwise direction (if viewing the pivot joint 134 from the bottom view in FIG. 13). Moving the pivotal member 110 in the counter-clockwise direction will cause the first roller 136 and the second roller 138 of the roller support member 112 to move away from the microscope slide 32.

As mentioned above, the alignment mechanism 100 in FIG. 1 is used in transporting microscope slides 32 between the retrieval unit 50 and the placer unit 150. The use of the alignment mechanism 100 as an intermediate step enables accurate placing of microscope slides 32 on the platen 200. The accurate placement of microscope slides 32 allows denser packing of microscope slides 32 on the platen 200 and more accurate printing operations. Additional benefits include increase throughput of the microarray printer 30 by enabling printing of more microscope slides 32 during a single run and increase efficiency of post-processing since the genetic material are found in a well defined position on the microscope slides 32. Moreover, the alignment mechanism enables asynchronous operations between the retrieval unit 50 and the placer unit 150.

Placer Unit

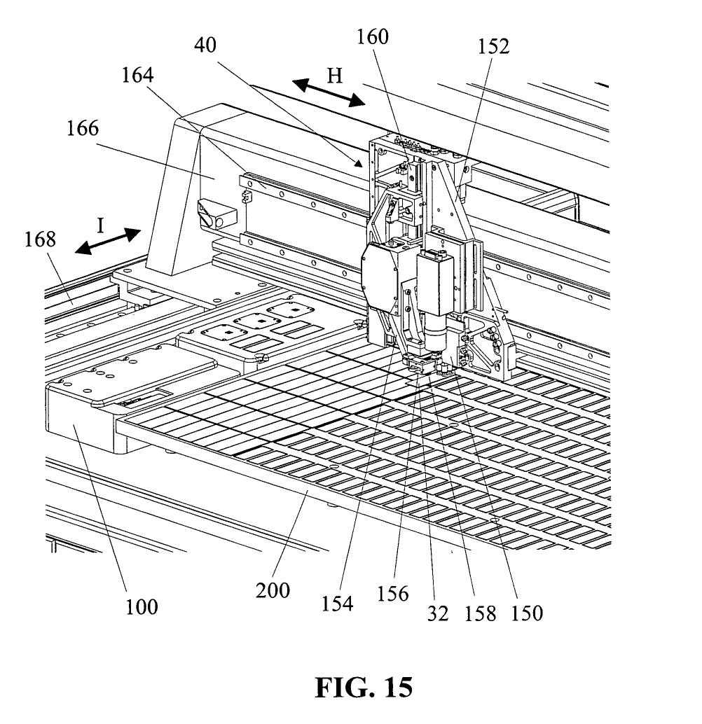

The purpose of the placer unit 150 is to position and remove microscope slides 32 to and from the platen 200. The placer unit 150 transports microscope slides 32 before and after printing operations. As shown in FIG. 15 and explained in more detail below, the placer unit 150 is coupled to the same gantry and carriage as the main printer arm of the printer device 40. Also explained in more detail below, the placer unit 150 has a mechanism to enable it to use the same arm used to perform printing operations.

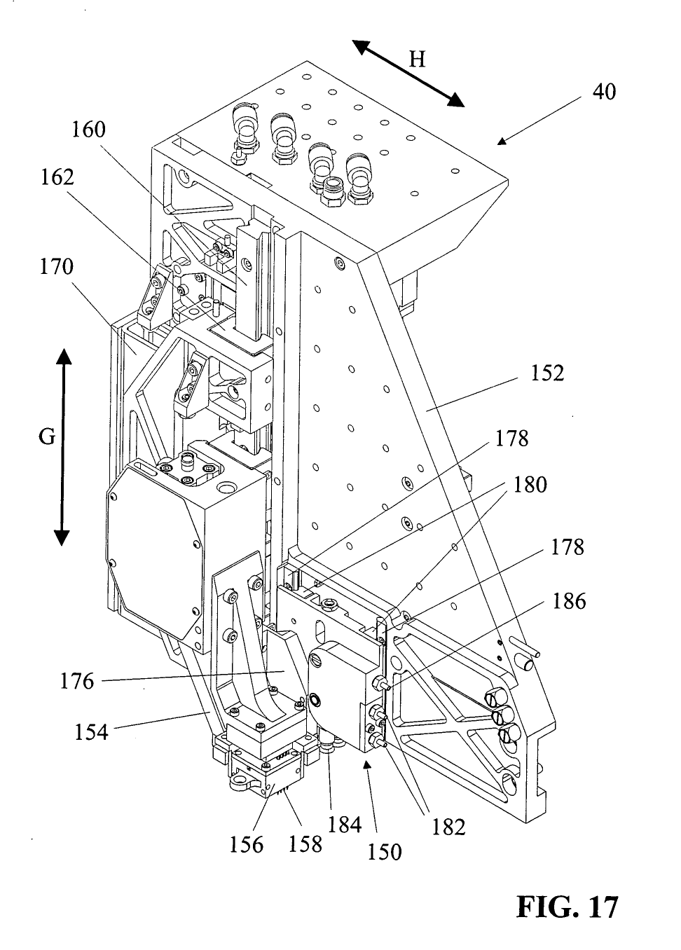

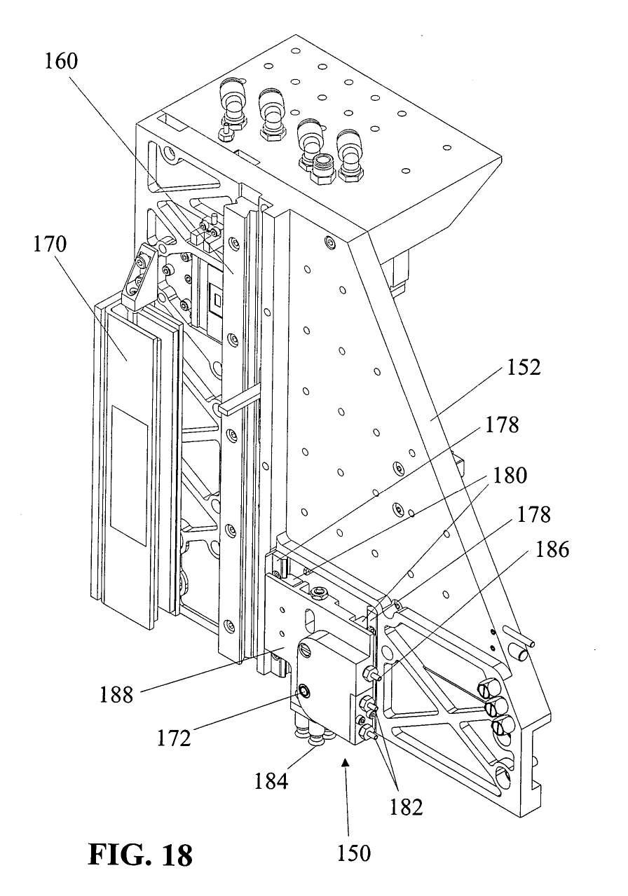

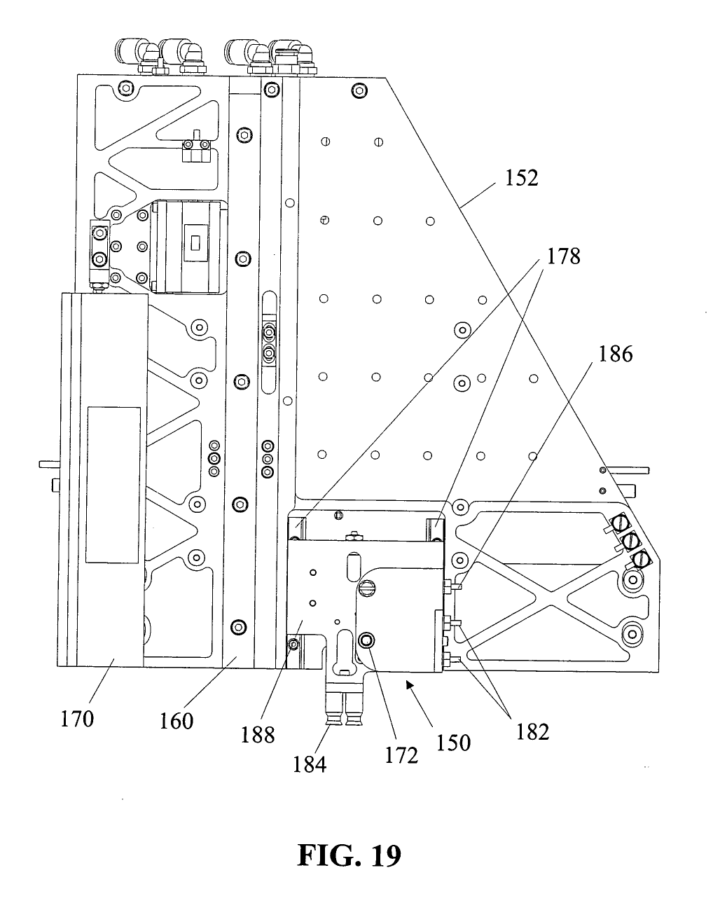

The main components of the printer device 40 include a printer carriage 152, a printer arm 154, a printer head 156, and printer pins 158. The printer pins 158 are coupled to the printer head 156 and perform the actual printing operations on the microscope slides 32. The printer head 156 is coupled to printer arm 154 and provides the support base for the printer pins 158. The printer arm 154 (along with printer head 156 and printer pins 158) is slidably attached to the printer carriage 152. The printer arm 154 slides in the direction shown by arrow G along a linear track 160 as shown in FIG. 17. In order to slide in direction G, in one embodiment, the printer arm 154 is attached to the linear track 160 via linear bearings 162. Moreover, a linear motor 170 may be used to translate the printer arm 154 along linear rail 160 as illustrated in FIGS. 17 and 18. Alternatively, it is contemplated that a pneumatic actuator may be used instead of a linear motor 170 to translate the printer arm 154 in the vertical direction G. In turn, the printer carriage 152 is mounted on a linear track 164 on a gantry 166. Mounting the printer carriage 152 on linear track 164 allows the printer carriage 152 to traverse along the gantry 166 in the direction as shown by arrow H in FIGS. 15, 16 and 17.

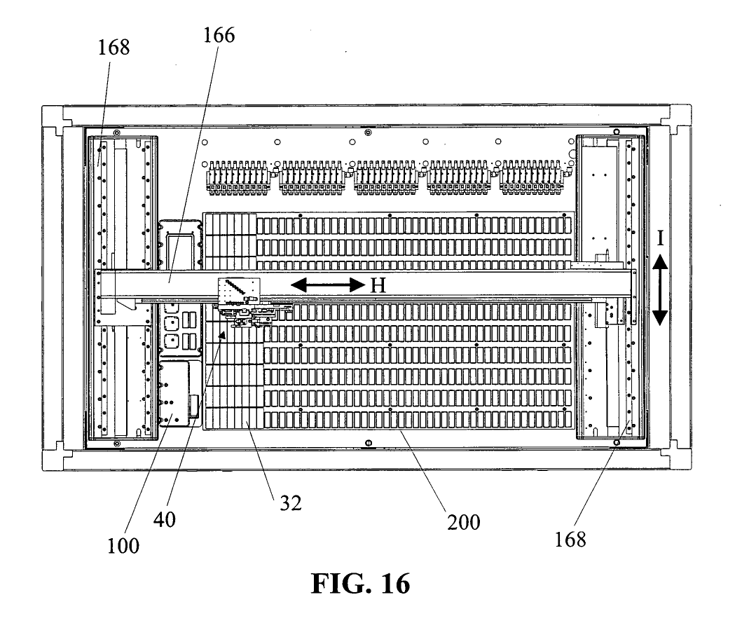

The gantry 166 enables the printer device 40 to move above each microscope slide 32 and perform precise printing operations. The printer arm 154 moves up and down in direction G to force the printer pins 158 against the top surface of the microscope slides 32. FIG. 16 shows a top view of the printer device 40 and gantry 166. The gantry 166 is located above the work surface of the platen 200. As described earlier, the printer carriage 152 translates along the gantry 166 in the direction shown by arrow H. Additionally, the gantry 166 is adapted to translate in a direction shown by arrow I as illustrated in FIGS. 15 and 16. The gantry 166 is allowed to move in direction I by mounting the gantry 166 on linear rails 168. An electric motor or pneumatic actuator may be used to translate the gantry 166 along linear rails 168. By allowing the printer carriage 152 to traverse in the directions shown by arrows H and I, the printer carriage 152 may position itself to any position above the platen 200 or above the alignment mechanism 100. Moreover, the movement of the printer carriage 152 and the gantry 166 are preferably controlled automatically by a computer system. To allow the computer system to recognize the precise location of the printer carriage 152, an encoder may be used to monitor its translation in the H and I directions. One suitable encoder is an RGH22 Analog Encoder by Renishaw Incorporated, 5277 Trillium Blvd., Hoffman Estates, Ill. 60192.

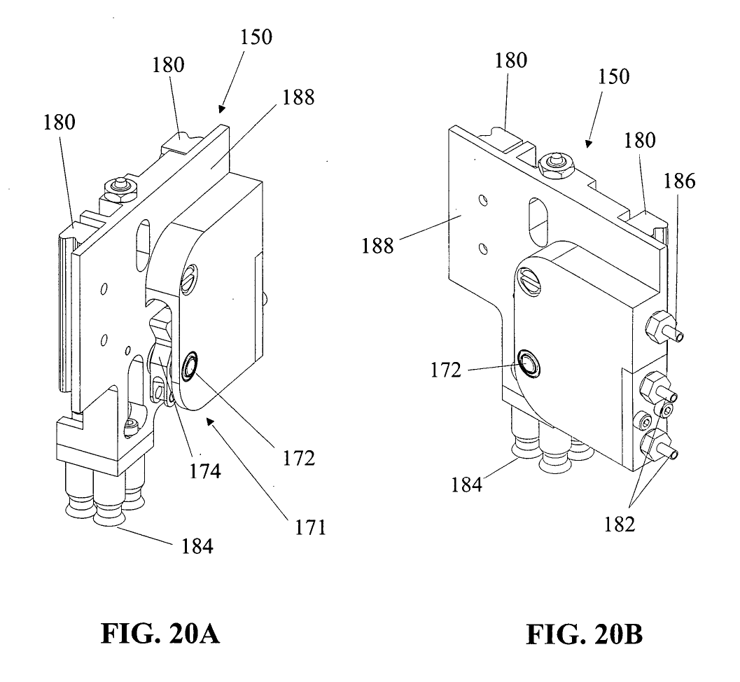

Referring to FIG. 17, the placer unit 150 is located in close proximity to the printer arm 154. During printing operations, the placer unit 150 is not coupled to the printer arm 154 and the printer arm 154 simply moves the printer head 156 and the printer pins 158 in the up and down direction. When the placer unit 150 needs to perform placement operations, however, a latching mechanism is used to couple the placer unit 150 to the printer arm 154. In one embodiment, the placer unit 150 has a support body 188, at least one suction cup or vacuum chuck 184 and a latching mechanism 171. The latching mechanism may include an actuator pin 172 and a latch 174. In the embodiment shown in FIG. 20A, the actuator pin 172 and latch 174 are mounted on the support body 188 of the placer unit 150. The latch 174 is rigidly attached to the actuator pin 172. When a microscope slide 32 needs to be transported to or from the platen 200, the latching mechanism is activated by rotating the actuator pin 172 in the counterclockwise direction by an actuator. The actuator (not shown) may be pneumatic or electric and drives the actuator pin 172. This extends the latch 174 and engages the latch 174 onto a latch receptor 176 is rigidly attached to the printer arm 154. After the latch 174 is locked on the latch receptor 176, the placer unit 150 is permitted to move in the up and down position by moving the printer arm 154 in the up and down direction. To permit the placer unit 150 to move up find down, the support body 188 of the placer unit 150 is mounted on a set of linear rails 178 via linear bearings 180.

If the actuator that drives the actuator pin 172 is pneumatic, two air interfaces 182 are provided on the placer unit 150 to extend and retract the latch 174. The two air interfaces 182 are connected by flexible tubing to a controlled air supply. One of the air interfaces 182 is used to extend and engage the latch 174 onto the latch receptor 176. This couples the placer unit 150 to the printer arm 154 and permits the placer unit 150 to position a microscope slide 32. The other air interface 182 is used to retract the latch 174 from the latch receptor 176. This disengages the placer unit 150 from the printer arm 154 and permits the printer arm 154 to perform printing operations. The actuator pin 172 is driven by an actuator that is controlled by a computer system.

The placer unit 150 has at least one suction cup or vacuum chuck 184 to pick up and hold a microscope slide 32. The vacuum chuck 184 is mounted on the support body 188 of the placer unit 150. To provide the necessary Suction to retain a microscope slide 32, the placer unit provides a vacuum interface 186 that is connected by flexible tubing to a vacuum supply. An electric or pneumatic valve (not shown) may be used to control the vacuum supply to the vacuum chuck 184. This valve may be controlled by a computer system to control the retention of a microscope slide 32 on the placer unit 150.

Enabling the placer unit 150 to use the same gantry 166, printer carriage 152 and printer arm 154 provides several benefits. First, redundant structures are eliminated reducing space and cost. The actuation hardware and control system for the printer device 40 will usually exist for a microarray printer 30 to perform printing operations. By coupling the placer unit 150 to the same structure, there is no need to introduce a second system for transporting microscope slides 32 to and from the platen 200. Second, the placer unit 150 benefits from the highly precise restrictions of the printer device 40 and gantry 166. In most systems, the printer device 40 must be accurate within 1 micrometer. By coupling the placer unit 150 to the same gantry 166, the placer unit 150 will be extremely accurate. The accuracy of the printer device 40 and placer unit 150 will be a function of the type of encoder used to locate the translation of the printer carriage 152 described earlier.

Platen

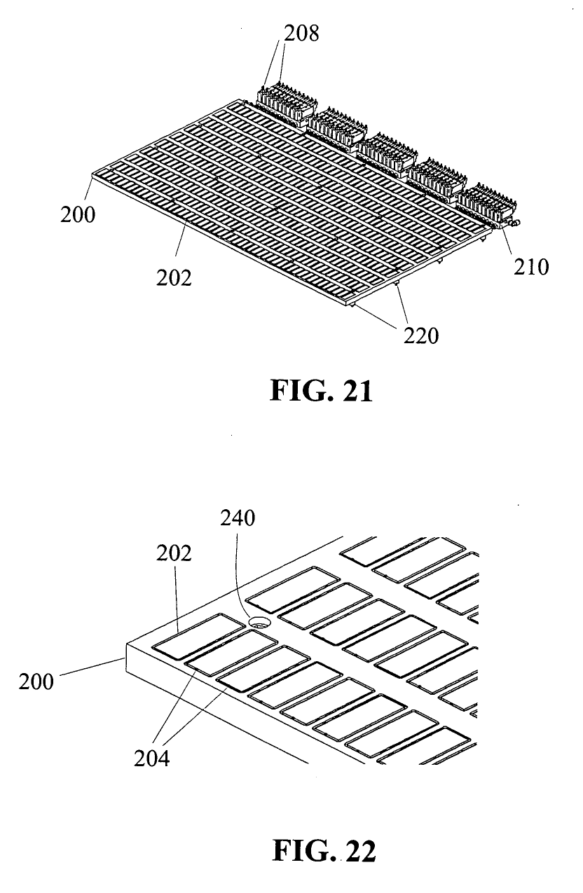

Referring to FIG. 21, the platen 200 acts as a worktable and provides a planar surface for the printer device 40 to perform printing operations on the microscope slides 32. The platen 200 is preferably made out of aluminum With a fine finish. The platen 200 has grooves 202 on its work surface. The grooves are preferably in the shape of a rectangle but may be of other shapes such as circular, oval or square. Inside each groove is a small restricting orifice 204 as illustrated in FIG. 22. The embodiment shown in FIG. 21 provides ten (10) rows and fifty (50) columns of rectangular grooves 202. This enables five hundred (500) microscope slides 32 to be placed on the platen 200 at one time. The present invention is not limited to these numbers and, having the benefit of this specification, a person skilled in the art would realize that a larger or smaller platen 200 could be used.

The grooves 202 are sized to be smaller then the rectangular shape of a microscope slide 32. The grooves 202 and restricting orifice 204 work together to hold each microscope slide 32 in place. The bottom side of each restricting orifice 204 has a pathway to a vacuum source. When a microscope slide 32 is place over the groove 32, a pressure differential is created between the bottom and top surfaces of the microscope slide 32. The bottom surfaces of the microscope slide 32 that extend over the groove 202 is pulled near vacuum pressure while the top surface of the microscope slide 32 is retained at atmospheric pressure. The pressure differential securely holds the microscope slide 32 on the work surface of the platen 200. In one embodiment, the groove 202 is rectangular in shape and has a depth in the range between about 0.01 inch to about 0.10 inch. In a preferred embodiment, the depth of the groove 202 is about 0.05 inch. The width of a rectangular groove 202 should range between about 0.01 inch to about 0.10 inch. In a preferred embodiment, the width of the groove 202 is about 0.05 inch.

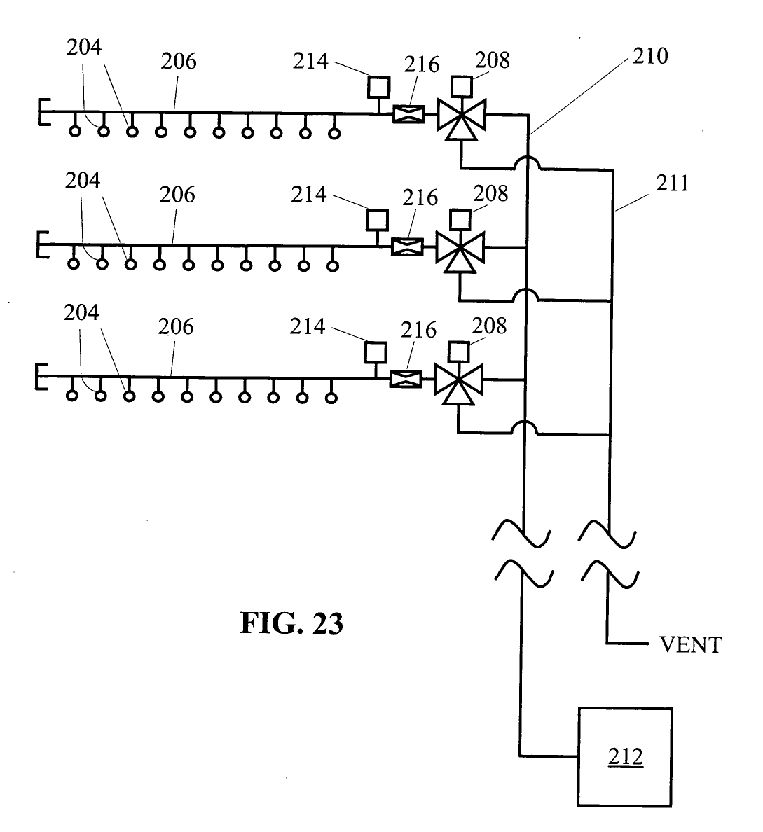

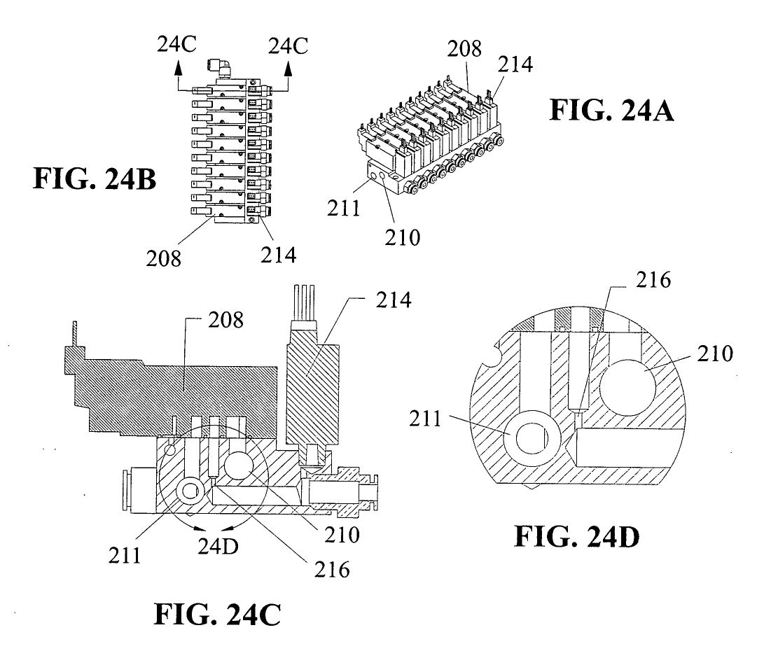

To provide adequate vacuum supply to each orifice 204, in one embodiment, the orifice 204 of each groove 202 along one column are tied to a single tube 206. For example, in the embodiment shown in FIG. 23, the orifices 204 of ten (10) rectangular grooves 202 are tied to a single source tube 206. In such a configuration, the platen 200 requires a separate source tube 206 for each column of grooves 202. As illustrated in FIG. 23, each source tube 206 is connected to a valve 208. The valve 208 may be electric, pneumatic or other means capable of being controlled by a computer system. The valve 208 is one of a plurality of valves that are connected to a vacuum manifold 210. The vacuum manifold 210 is connected to a vacuum source or pump 212. As shown in FIGS. 24A and 24B, the valves may be closely aligned to each other along the vacuum manifold 210.

The vacuum manifold 210 provides a constant source of vacuum pressure to each source tube 206 when the valve 208 is in the open position. Referring to FIGS. 23 and 24C, a pressure sensor 214 is also located on the source tube side of each valve 208. The pressure sensor 214 is used to monitor the pressure in each source tube 206. A flow restrictor 216 is preferably used between the pressure sensor 214 and the valve 208 as illustrated in FIG. 24D. The flow restrictor 216 allows a more accurate reading of the pressure in the source tube 206 by the pressure sensor 214. In one embodiment, where a single column consists of ten (10) rectangular grooves, the diameter of the flow restrictor 216 should range between about 0.01 inch to about 0.10 inch. In a preferred embodiment, the diameter of flow restrictor 216 is about 0.05 inch.

The valves 208 may also be connected to a vent manifold 211 as shown in FIGS. 23 and 24. This allows the source tube 206 to vent to atmospheric pressure when a microscopes slide 32 needs to be removed from the platen 200.

The restricting orifices 204 must be appropriately sized to ensure that when valve 208 is open to vacuum that each orifice 204 provides enough suction to retain a single microscope slide 32. If the restricting orifices 204 are too large, the first microscope slide 32 placed in a selected column will not stay retained on the platen 200. Thus, the flow rate through each column's combined restricting orifices 204 must be low enough so that the pressure within each source tube 206 does not equalize to atmospheric pressure. In one embodiment, where a single column consists of ten (10) rectangular grooves, the diameter of each orifice 204 should range from about 0.010 inch to about 0.100 inch. In a preferred embodiment, the diameter of each orifice 204 is about 0.024 inch.

The pressure sensor 214 may be monitored by a computer system to determine the number of microscope slides 32 that are in position on the platen 200 along any single column. For example, as each restricting orifice 204 is blocked by positioning a microscope slide 32 over the corresponding groove 202, the pressure within the source tube 206 will drop. Once a complete column of microscope slides 32 are in place, each restricting orifice 204 will be blocked creating a vacuum within source tube 206. The computer system may be programmed to monitor each pressure sensor 214 to determine whether microscope slides 32 are being added or removed from the platen 200.

It is important for the platen 200 to be extremely planar. Height consistency of individual microscope slides 32 yields consistent impact printing. In addition, any variation in planarity must be absorbed into the printing clearance height. In other words, the printer pens 158 of the printing device 40 must clear the highest microscope slide 32 on the platen 200. Minimizing the clearance height between microscope slides 32 increases printing rates. Moreover, the higher the clearance height, the longer it takes for the printing pens to traverse up and down.

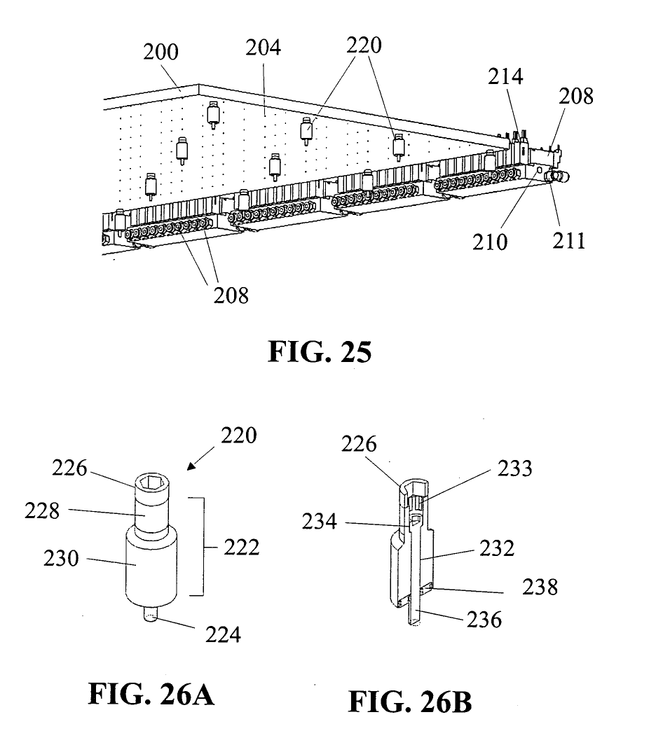

An alignment system was developed to adequately align and lock the work surface of the platen 200. The alignment system also enables a user to compensate for manufacturing variations, temperature variations, vibration variations, sags due to gravity, or any other phenomena that may pull the platen 200 out of planarity. To align the platen 200, the platen 200 uses a plurality of positioning devices 220 as illustrated in FIG. 25. For sake of clarity, FIG. 25 does not show the source tubes 206 that connect the orifices 204 to the valves 208. In the embodiment shown in FIG. 20, twenty (20) positioning device 220 are used at various locations on the platen 200 although the present invention is not limited to that number. The positioning devices 220 are preferably equally spaced throughout the platen 200 to provide the maximum flexibility in aligning the platen 200.

Referring to FIGS. 26A and 26B, the positioning device 220 comprises of three main components, an outer adjustment member 222, an inner bolt 224, and a locking member 226. The outer adjustment member 222 has an outer threaded portion 228, a main body portion 230, and a cylindrical bore 232. The platen 200 is attached to the outer threaded portion 228 of the outer adjustment member 222. The platen 200 has threaded circular holes 240 that receive the threaded portion 228 of the outer adjustment member 222. The positioning devices 220 are mounted underneath the platen 200 as illustrated in FIG. 25. The top portion 233 of the cylindrical bore 232 is shaped to enable a driver device (not shown) to rotate the outer adjustment member 222 in the threaded circular hole 240. In one embodiment, the top portion 233 of the cylindrical bore 232 is in the shape of a hexagon. As the outer adjustment member 222 is rotated, the threaded portion 228 of the outer adjustment member 222 forces the platen 200 in either the up or down direction.

The inner bolt 224 has a head portion 234 and a shaft portion 236. The shaft portion 236 of the inner bolt 224 is slidably received in a bottom portion of the bore 232 of the outer adjustment member 222. Moreover, the outer adjustment member 222 is pivotally mounted to the inner bolt 224 such that the outer adjustment member 222 is allowed to rotate about the inner bolt 224. In one embodiment, the cylindrical bore 232 of the outer adjustment member 222 has bearings 238 that wrap around the inner bolt 224. The inner bolt 224 is rigidly attached to a fixed surface of the microarray printer 30. The head portion 234 of the inner bolt 224 is shaped to enable a driver device (not shown) to rotate the inner bolt 224 in order to secure the bolt to a fixed surface of the microarray printer 30. In one embodiment, the head portion 234 of the inner bolt 224 is in the shape of a hexagon.

The locking member 226 is used to lock the outer adjustment member 222 into position after the platen 200 is at the correct level. The locking member 226 is cylindrical in shape. The outer surface of the locking member 226 has threads that engage with the threaded circular holes 240. The locking member 226 has a shaped bore 242 to enable a driver device (not shown) to rotate the locking member 226 in the threaded circular hole 240. In one embodiment, the bore 242 of the locking member is in the shape of a hexagon. The locking member 226 serves as a lock nut after the platen 200 is level.

It is apparent from the above description that different sized driver devices may be used to perform various functions of the positioning device 220. For example, one type of driver device may be used to lock the inner bolt 224 into a fixed surface of the microarray printer 30. Another type of driver device may be used to adjust the level of the platen 220 by rotating the outer adjustment member 222. A further type of driver device may be used to lock the outer adjustment member 222 into position by rotating the locking member 226 within the threaded hole 240 of the platen 200.

While the present invention has been described with reference to one or more particular embodiments, those skilled in the art will recognize that many changes may be made thereto without departing from the spirit and scope of the present invention. Each of these embodiments and obvious variations thereof is contemplated as falling, within the spirit and scope of the claimed invention, which is set forth in the following claims.

What is claimed is:

1. An automated microarray printer machine having the capability of automatically transporting a plurality of microarray workpieces before or after printing operations by a printer device of said microarray printer machine, said automated microarray printer machine comprising:
a storage unit for storing said plurality of microarray workpieces;
a workstation; and
a retrieval mechanism for retrieving one of said plurality of microarray workpieces from said storage unit and presenting said microarray workpiece to said workstation.

2. The automated microarray printer machine of claim 1, wherein said storage unit has a storage frame, a storage rack and a first means for translating said storage rack in a first plane, said storage rack adapted to store said plurality of microarray workpieces, said storage rack mounted slidably to said storage frame in said first plane.

3. The automated microarray printer machine of claim 1, wherein said workstation is an alignment mechanism.

4. The automated microarray printer machine of claim 1, wherein said retrieval mechanism has a loader frame, a loader arm and a second means for translating said loader arm in a second plane, said loader arm mounted slidably to said loader frame in the second plane, said loader arm capable of accessing one of said plurality of microarray workpieces from said storage unit, said loader arm having a vacuum chuck for retaining said one of said plurality of microarray workpieces on said loader arm.

5. The automated microarray printer machine of claim 1, wherein said plurality of microarray workpieces are a plurality of microscope slides.

6. A retrieval unit for a microarray processing device to transport a workpiece before or after processing operations by said microarray processing device, said retrieval unit comprising:
a storage unit having a storage frame, a storage rack and a first motor, said storage rack adapted to store said workpiece, said storage rack mounted slidably to said storage frame in a first plane, said first motor adapted to slide said storage rack in said first plane; and
a lifter unit having a loader frame, a loader arm and a second motor, said loader arm mounted slidably to said loader frame in a second plane, said second motor adapted to slide said loader arm in the second plane, said loader arm capable of accessing said workpiece from said storage rack, said loader arm having a vacuum chuck for retaining said workpiece on said loader arm.

7. The retrieval unit of claim 6, wherein said microarray processing device is a microarray printer.

8. The retrieval unit of claim 6, wherein said workpiece is a microscope slide.

9. The retrieval unit of claim 6, wherein said storage rack has a plurality of slots adapted to receive a plurality of workpiece containers, each workpiece container capable of retaining a plurality of workpieces.

10. The retrieval unit of claim 9, wherein each of said plurality of slots has a sensor to determine whether one of said plurality of workpiece containers is contained within said slot.

11. The retrieval unit of claim 6, wherein said loader arm has a third motor adapted to slide said vacuum chuck in a third plane.

12. The retrieval unit of claim 6, wherein said first motor and said second motor are controlled by a computer.

13. The retrieval unit of claim 11, wherein said first motor, said second motor, and said third motor are controlled by a computer.

14. The retrieval unit of claim 6, wherein said loader arm is capable of transporting a workpiece from said storage rack to a workstation.

15. The retrieval unit of claim 14, wherein said workstation is an alignment mechanism.

16. An automated microarray printer for performing printing operations on a workpiece, said automated microarray printer having a computer to control the transportation of the workpiece before said printing operations, said microarray printer comprising:
a retrieval unit capable of retrieving said workpiece for said printing operations, said retrieval unit having a storage unit and a lifter unit;
said storage unit having a storage frame, a storage rack and a first motor, said storage rack mounted slidably to said storage frame in a first plane, said first motor controlled by said computer and adapted to slide said storage rack in said first plane; and
said lifter unit having a loader frame, a loader arm and a second motor, said loader arm mounted slidably to said loader frame in a second plane, said second motor controlled by said computer and adapted to slide said loader arm in the second plane, said loader arm capable of accessing said workpiece from said storage unit, said loader arm having a vacuum chuck to hold said workpiece on said loader arm.

17. The microarray printer of claim 16, wherein said workpiece is a microscope slide.

18. The microarray printer of claim 16, wherein said storage rack has a plurality of slots adapted to receive a plurality of workpiece containers, each workpiece container capable of retaining a plurality of workpieces.

19. The microarray printer of claim 18, wherein each of said plurality of slots has a sensor to determine whether one of said plurality of workpiece containers is contained within said slot.

20. The microarray printer of claim 16, wherein said loader arm has a third motor controlled by said computer and adapted to slide said vacuum chuck in a third plane.

21. The retrieval unit of claim 16, wherein said loader arm is capable of transporting a workpiece from said storage rack to a workstation.

22. The retrieval unit of claim 21, wherein said workstation is an alignment mechanism.

23. A method within a microarray printer machine for retrieving a workpiece in a storage rack and transporting said workpiece to a workstation, said microarray printer machine having a loader arm with a vacuum chuck, said method comprising:
determining the location of the workpiece in said storage rack;
moving said loader arm in close proximity to said workpiece in said storage rack;
extending said vacuum chuck under said workpiece;
activating said vacuum chuck to hold said workpiece on said loader arm; and
moving said loader arm and workpiece to said workstation.

24. The method of claim 23, wherein said workpiece is a microscope slide.

25. The method of claim 23, wherein said step of determining the location of the workpiece in said storage rack includes receiving a set of coordinates for the workpiece from a computer.

26. The method of claim 23, wherein said step of moving said loader arm in close proximity to said workpiece includes activating at least one motor that slides said loader arm in at least one plane.

27. The method of claim 23, wherein said step of moving said loader arm in close proximity to said workpiece includes activating at least one motor that slides said storage rack in at least one plane.

28. The method of claim 23, wherein said step of extending said vacuum chuck under said workpiece includes activating at least one motor that slides said vacuum chuck in at least one plane.

29. The method of claim 23, wherein said step of activating said vacuum chuck includes opening a valve to expose said vacuum chuck to a vacuum source.

30. The method of claim 23, wherein said step of moving said loader arm and workpiece to said workstation includes activating at least one motor that slides said loader arm in at least one plane.

31. The method of claim 23, wherein said workstation is an alignment mechanism for aligning the workpiece.

32. A method within a microarray printer for retrieving a workpiece in a storage rack and transporting said workpiece to a workstation, said microarray printer having a first motor to slide said storage rack in a first plane, said microarray printer having a second motor to slide a loader arm in a second plane, said loader arm having a vacuum chuck, said method comprising:
determining the location of the workpiece in said storage rack;
moving said loader arm in close proximity to said workpiece in said storage rack by activating said first motor and said second motor;
extending said vacuum chuck toward said workpiece;
activating said vacuum chuck to hold said workpiece on said loader arm; and
moving said loader arm and workpiece to said workstation by activating said first motor.

33. The method of claim 32, wherein said workpiece is a microscope slide.

34. The method of claim 32, wherein said step of determining the location of the workpiece in said storage rack includes receiving a set of coordinates for the workpiece from a computer.

35. The method of claim 32, wherein said step of extending said vacuum chuck under said workpiece includes activating at least one motor that slides said vacuum chuck in at least one plane.

36. The method of claim 32, wherein said step of activating said vacuum chuck includes opening a valve to expose said vacuum chuck to a vacuum source.

37. The method of claim 32, wherein said workstation is an alignment mechanism for aligning the workpiece.

38. A retrieval unit for a microarray processing device to transport a workpiece before or after processing operations by said microarray processing device, said retrieval unit comprising:
a storage unit having a storage frame, a storage rack and a first means for translating said storage rack in a first plane, said storage rack adapted to store said workpiece, said storage rack mounted slidably to said storage frame in said first plane; and
a lifter unit having a loader frame, a loader arm and a second means for translating said loader arm in a second plane, said loader arm mounted slidably to said loader frame in the second plane, said loader arm capable of accessing said workpiece from said storage rack, said loader arm having a vacuum chuck for retaining said workpiece on said loader arm.

39. The retrieval unit of claim 38, wherein said microarray processing device is a microarray printer device.

40. The retrieval unit of claim 38, wherein said workpiece is a microscope slide.

41. The retrieval unit of claim 38, wherein said storage rack has a plurality of slots adapted to receive a plurality of workpiece containers, each workpiece container capable of retaining a plurality of workpieces.

42. The retrieval unit of claim 41, wherein each of said plurality of slots has a sensor to determine whether one of said plurality of workpiece containers is contained within said slot.

43. The retrieval unit of claim 38, wherein said loader arm has a third means for translating said vacuum chuck in a third plane.

44. The retrieval unit of claim 38, wherein said loader arm is capable of transporting a workpiece from said storage rack to a workstation.

45. The retrieval unit of claim 44, wherein said workstation is an alignment mechanism.
